(19) United States
(12) Patent Application Publication
(10) Pub. No.: US 2012/0309785 A1
(43) Pub. Date: Dec. 6, 2012

(54) Use of Cysteamine in Treating Parkinson's Disease

(76) Inventor: Bill Piu Chan, Beijing (CN)

(21) Appl. No.: 13/311,098

(22) Filed: Dec. 5, 2011

Related U.S. Application Data
(60) Provisional application No. 61/419,521, filed on Dec. 3, 2010.
(60) Provisional application No. 61/437,260, filed on Jan. 28, 2011.

(51) Int. Cl. A61K 31/131; A61K 31/13; A61K 31/198; A61K 31/4453; A61K 31/137; A61P 25/16; A61K 31/46

(52) U.S. Cl. 514/304; 514/665; 514/662; 514/317; 514/654; 514/567

(57) ABSTRACT

The subject invention provides materials and methods for treating neurodegenerative diseases. In one embodiment of the invention, a cysteamine compound is administered to a patient to treat Parkinson's Disease and/or complications associated with Parkinson's Disease. In another embodiment, a cysteamine compound is administered to a patient to prevent the onset of Parkinson's Disease in an at-risk patient and/or treat or prevent the onset of Parkinson's Disease-associated symptoms.

### CROSS-REFERENCE TO A RELATED APPLICATION

This application claims the benefit of U.S. Provisional Application Ser. No. 61/419,521, filed Dec. 3, 2010 and U.S. Provisional Application Ser. No. 61/437,260, filed Jan. 28, 2011, both of which are incorporated herein by reference in their entirety.

### BACKGROUND

Parkinsons Disease (PD) is a disturbance of voluntary movement in which muscles become stiff and sluggish, movement becomes clumsy and difficult, and uncontrollable rhythmic twitching of groups of muscles produces characteristic shaking or tremor. It is a progressive neurodegenerative disease characterized by the loss of dopaminergic neurons and, consequently, depletion of dopamine (DA) in its striatal projections (Dawson et al., “Molecular pathways of neurodegeneration in Parkinson's disease,” Science, 302:819-822 (2003)). The absence of adequate release of the chemical transmitter dopamine during neuronal activity thereby leads to the Parkinsonian symptomatology.

Although the etiology of PD remains unknown (Wolters, “PD-related psychosis: pathophysiology with therapeutical strategies,”J Neural Transm Suppl, 71:31-37 (2006)), mitochondrial dysfunction, oxidative stress and other mechanisms are believed to be pivotal factors involved in the death of dopaminergic neurons. In particular, oxidative stress and mitochondrial dysfunctions leading to excessive generation of reactive oxygen species (ROS) are known to be crucial factors in the initiation and progression of various neurodegenerative disorders (Wong et al., “Oxidative stress induced by MPTP and MPP(+): selective vulnerability of cultured mouse astrocytes,” Brain Res, 836:237-244 (1999); Albanese et al., “Chronic administration of 1-methyl-4-phenyl-1,2,3,6-tetrahydropyridine to monkeys: behavioural, morphological and biochemical correlates,” Neuroscience, 55:823-832 (1993)). Continuous burden of ROS ultimately causes cells to develop an oxidative condition (Nordberg et al., “Reactive oxygen species, antioxidants, and the mammalian thioredoxin system,” Free Radic Biol Med, 31:1287-1312 (2001)). For example, decreases in activities of mitochondrial complex I, superoxide dismutase (SOD) and glutathione level, and increases in malondialdehyde (MDA) which catalyzes the Fenton reaction, have been observed in idiopathic PD (Sharpe et al., “Oxidation of nitric oxide by oxomanganese-salen complexes: a new mechanism for cellular protection by superoxide dismutase/catalase mimetics,” Biochem J, 366:97-107 (2002); Shadrina et al., “Mitochondrial dysfunction and oxidative damages in the molecular pathology of Parkinson's disease,” Mol Biol (Mosk), 42:809-819 (2008); Bharath et al., “Glutathione depletion in a midbrain-derived immortalized dopaminergic cell line results in limited tyrosine nitration of mitochondrial complex I subunits: implications for Parkinson's disease,” Antioxid Redox Signal, 7:900-7910 (2005)). Furthermore, decreased release of the brain-derived neurotrophic factor (BDNF) observed in the striatum, also contribute to the exacerbation of PD (Kuipers et al., “Brain-derived neurotrophic factor mechanisms and function in adult synaptic plasticity: new insights and implications for therapy,” Curr Opin Drug Discov Devel. 9:580-586 (2006)).

Currently, the most widely used treatment for Parkinsonism is administration of L-DOPA, a precursor of dopamine that acts indirectly by replacing the missing dopamine. However, disadvantages are associated with the use of L-DOPA, for example, patients often suffer from side-effects such as dyskinesia and on-off effects, and it is necessary to administer L-DOPA in conjunction with a peripheral dopa-decarboxylase inhibitor such as carbidopa or benzaseride. These inhibitors prevent the peripheral degradation of levodopa to dopamine, thus enabling more drug to enter the brain and limiting peripheral side-effects. Such treatment improves quality of life for patients but does not halt disease progression. Furthermore, such treatment is associated with a number of adverse effects including nausea, vomiting, abdominal distension and psychiatric side-effects (for example, toxic confusional states, paranoia and hallucinations).

An alternative form of therapy is to administer postsynaptic dopamine agonists, for example ergot alkaloids such as bromocriptine. This approach, however, is also associated with side-effects. For example, patients receiving bromocriptine often experience dyskinesia psychiatric problems, and in a small number of cases experience vasopastic phenomena and angina. In addition, bromocriptine causes psychiatric side-effects such as hallucinations.

These and other existing therapies for PD are mainly for symptom management and, so far, no treatment is available for attenuating the progression of the disease. The ultimate cure for the disease requires approaches to protect the DA neurons against damage and to halt or slow the continuous functional decline of these cells at early pathological stages. One such approach is to recover the mitochondrial function and increase the reductase activities by anti-oxidant reagents that allow for elimination of excessive ROS and inhibition of oxidative cell stress.

A number of such reagents have been tested for their potential in protecting the dopaminergic neurons against oxidative cell stress in rodent models of PD. For example, crocetin was proved to ameliorate symptoms in a hemi-parkinsonian rat model by increasing SOD activity and the GSH level (Ahmad et al., “Neuroprotection by crocetin in a hemi-parkinsonian rat model,” Pharmacol Biochem Behav, 81:805-813 (2005)). In addition, one study suggested that coenzyme Q10 reduced the depletion of TH-positive DA neurons in a chronic dosing model of PD induced by 1-methyl-4-phenylpyridinium (MPP⁺) (Cleren et al., “Therapeutic effects of coenzyme Q10 (CoQ10) and reduced CoQ10 in the MPTP model of Parkinsonism,” J Neurochem, 104:1613-1621 (2008)). Other anti-oxidant reagents with similar properties include N-acetylcysteine (Bagh et al., “Quinone and oxyradical scavenging properties of N-acetylcysteine prevent dopamine mediated inhibition of Na⁺, K⁺-ATPase and mitochondrial electron transport chain activity in rat brain: implications in the neuroprotective therapy of Parkinson's disease,” Free Radic Res, 42:574-581 (2008)), polyhydroxylated fullerene derivative such as C(60)(OH) (Cai et al., “Polyhydroxylated fullerene derivative C(60)(OH)(24) prevents mitochondrial dysfunction and oxidative damage in an MPP(+)-induced cellular model of Parkinson's disease,” J Neurosci Res, 86:3622-3634 (2008)), and herbal extracts such as green tea polyphenols or catechins (Chung et al., “Epigallocatechin gallate (EGCG) potentiates the cytotoxicity of rotenone in neuroblastoma SH-SY5Y cells,” Brain Res, 1176:133-142 (2007); Wang et al., “Rg1 reduces nigral iron levels of MPTP-treated C57BL6 mice by regulating certain iron transport proteins,” Neurochem Int, 54:43-48 (2009); Rojas et al., “Effect of EGb761 supplementation on the content of copper in mouse brain in an animal model of Parkinson's disease,” Nutrition, 25:482-485 (2009)).

BDNF, one of the key neurotrophic factors, is expressed by dopamine neurons in both the substantia nigra and the ventral tegmental area and plays a key role in the survival and differentiation of midbrain dopaminergic neurons (Tsai, “Attention-deficit hyperactivity disorder may be associated with decreased central brain-derived neurotrophic factor activity: clinical and therapeutic implications,” Med Hypotheses. 68:896-899 (2007)). Studies have shown that BDNF is required to maintain the proper number of dopaminergic neurons in the substantia nigra and decreased midbrain BDNF activity may cause DA dysfunction (Zuccato et al., “Role of brain-derived neurotrophic factor in Huntington's disease,” Prog Neurobiol. 81:294-330 (2007)).

With patients diagnosed with Huntington's Disease, mechanisms underlying the upregulation of BDNF by cysteamine might be similar to that suggested by Goggi (Goggi et al., “Signalling pathways involved in the short-term potentiation of dopamine release by BDNF,” Brain Res. 968:156-161 (2003)) and Borrell (Borrell et al., “Cystamine and cysteamine increase brain levels of BDNF in Huntington disease via HSJ1b and transglutaminase,” J Clin Invest. 116, 1410-1424 (2006)), where cysteamine can directly enhance BDNF secretion from the Golgi apparatus through a heat shock DnaJ-containing protein 1,b (HSJ1b)-dependent mechanism involving transglutaminase inhibition. BDNF, in turn, can modulate the release of dopamine through activation of the TrkB receptors (Narita et al., “Implication of brain-derived neurotrophic factor in the release of dopamine and dopamine-related behaviors induced by methamphetamine,” Neuroscience. 119:767-775 (2003)). Moreover, it has been reported that the increased BDNF levels in brain and serum were still detectable 12 weeks after continuous cysteamine treatment, indicating that the efficacy of a treatment course of repeated cysteamine is unlikely to decrease with time (Narita et al., supra 2003).

Cysteamine prevents lipid peroxidation and protein carbonylation, and improves activities of superoxide dismutase, glutathione peroxidase and catalase (Dubinsky et al., “CYTE-I-HD: phase I dose finding and tolerability study of cysteamine (Cystagon) in Huntington's disease,” Mov Disord. 21:530-533 (2006); Kessler et al., “Antioxidant effect of cysteamine in brain cortex of young rats,” Neurochem Res. 33:737-744 (2008); and Kessler et al., “Effects of cysteamine on oxidative status in cerebral cortex of rats,” Metab Brain Dis. 23:81-93 (2008)). Cysteamine also normalizes creatine kinase and pyruvate kinase which are crucial for energy homeostasis and anti-oxidant defenses in cystinosis (Rech et al., “Cysteamine prevents inhibition of thiol-containing enzymes caused by cystine or cystine dimethylester loading in rat brain cortex,” Metab Brain Dis. 23:133-145 (2008)). Recent studies found that cysteamine and cystamine have neuroprotective effects in a mouse model of Huntington's disease (HD) via inhibiting the transglutaminase activity and enhancing the BDNF level (Karpuj et al., “Prolonged survival and decreased abnormal movements in transgenic model of Huntington disease, with administration of the transglutaminase inhibitor cystamine,” Nat Med. 8:143-149 (2002)). In vitro studies suggested that cystamine and cysteamine can prevent the depolarization of mitochondria membrane and increase cellular GSH levels in the Huntingtin-mutant mice (Mao et al., “Cystamine and cysteamine prevent 3-NP-induced mitochondrial depolarization of Huntington's disease knock-in striatal cells,” Eur J Neurosci. 23:1701-1710 (2006)). However, such effects have not been tested either experimentally or clinically in PD.

Despite the availability of certain potential reagents in protecting the dopaminergic neurons against oxidative cell stress, there remains a great continuing need for the provision of effective, safe medicaments for the treatment of PD.

### BRIEF SUMMARY OF THE INVENTION

The subject invention provides materials and methods for treating and/or attenuating the progression of Parkinsons Disease (PD). Specifically exemplified herein is the use of cysteamine, or a salt thereof, to treat and/or attenuate the progression of PD. For example, at least partial recovery of mitochondrial function, reduction in ROSs, and inhibition of oxidative stress in neurons can be achieved through administration, according to the subject invention, of cysteamine or a salt thereof (such as cysteamine hydrochloride).

In accordance with the subject invention, the administration of a cysteamine compound to a patient can delay, or otherwise inhibit, the development of PD. In one embodiment, a cysteamine compound is administered to a patient who has no observable symptoms of PD but has been determined to be susceptible to developing PD (hereinafter such a patient is referred to as an “at-risk patient”). In a specific embodiment, a patient is assessed to identify the risk of developing PD prior to the administration of a cysteamine compound.

In preferred embodiments of the subject invention, a cysteamine compound is administered to a patient prior to, or after, diagnosis of Parkinson's Disease to treat, attenuate the progression of, or ameliorate symptoms associated with PD.

According to the present invention, the administration of a cysteamine compound to a patient can ameliorate the loss of dopaminergic neurons and/or reduced striatal dopamine concentrations. In addition, administration of a cysteamine compound to a patient suppresses the production of pro-oxidants, such as reactive oxygen species (ROS) and malondialdehyde (MDA), which have been observed in idiopathic PD. Further, the methods of the subject invention can be used to restore secretion of the brain-derived neurotrophic factor (BDNF) by neurons derived from substantia nigra pars compact (SNpc).

In accordance with the subject invention, the daily dosage amount of a cysteamine compound administered to a patient diagnosed with PD or suffering from symptoms associated with PD is about 0.1 mg to about 1,000 mg/kg of patient body weight (BW) of a cysteamine compound.

Preferably, a daily dose of less than about 30 mg/kg of BW of cysteamine, is administered to a patient to treat PD in accordance with the present invention. More preferably, a low daily dose of 20 mg/kg of BW or less of cysteamine is administered to a patient to treat PD in accordance with the subject invention.

A cysteamine compound can be administered alone or concurrently with other known therapeutic agents and methods for treating PD. Contemplated agents and methods include, without limitation, general wellness maintenance, physiotherapy, exercise, nutrition, administration of agents such as catechol-O-methyl transferase (COMT) inhibitors (such as entacapone, tolcapone, nitecapone); dopamine agonists (such as pramipexole, ropinerole, bromocriptine, and pergolide); amantadine; benztropine; trihexyphenydil; deprenyl; and L-DOPA alone or in combination with carbidopa or benserazide.

### BRIEF DESCRIPTION OF THE DRAWINGS

FIG. 1 is a graphical illustration of the ability of cysteamine to increase the number of TH-immunoresponsive (TH-IR) neurons in the substantia nigra of MPTP-treated mice. TH-IR neuronal counts were presented as mean±SD and their numbers were compared among different treatment groups.

FIG. 2 is a graphical illustration of cysteamine's ability to protect against MPTP-induced decrease in striatal dopamine (DA) concentrations in mice. Concentrations of DA (ng/mg protein, mean±SD) in the corpus striatum tissues derived from mice receiving different treatments were measured by high performance liquid chromatography (HPLC) and compared among treatment groups. P values are as indicated on the figure.

FIG. 3 is a graphical illustration of cysteamine's ability to protect against MPTP-induced decrease of glutathione (GSH) levels in the SNpc of mice. GSH concentration (nmol TNB/mg protein, mean±SD) of the isolated cells of substantia nigra from mice with different treatments was measured using glutathione reductase and compared among treatment groups. P values are as indicated on the figure.

FIG. 4 is a graphical illustration of cysteamine's ability to attenuate MPTP-induced decrease in the content of brain-derived neurotrophic factor (BDNF) in the SNpc of mice. BDNF concentrations (pg/mg protein, mean±SD) were measured by an ABC-ELISA method for the isolated cells of substantia nigra from mice with different treatment and compared among treatment groups. P values are as indicated on the figure.

FIGS. 5A-H are images of coronal sections of substantia nigra (SN) derived from mice with different treatment conditions that were stained with anti-TH monoclonal (3D5) antibody, where TH-IR nigral neurons were visualized microscopically (40×). FIG. 5A: Saline control; FIG. 5B: MPTP alone; FIG. 5C: Cysteamine 20 mg/kg/d alone; FIG. 5D: Cysteamine 75 mg/kg/d alone; FIG. 5E: Cysteamine 20 mg/kg/d pretreatment followed by MPTP; FIG. 5F: Simultaneous treatment of MPTP and cysteamine 20 mg/kg/d; FIG. 5G: Cysteamine 75 mg/kg/d treatment followed by MPTP; FIG. 5H: Simultaneous treatment of MPTP and Cysteamine 75 mg/kg/d.

FIG. 6 illustrates images of coronal sections of substantia nigra (SN) derived from mice with different treatment conditions that were stained with anti-TU monoclonal (3D5) antibody, where TH-IR nigral neurons were visualized microscopically (40×).

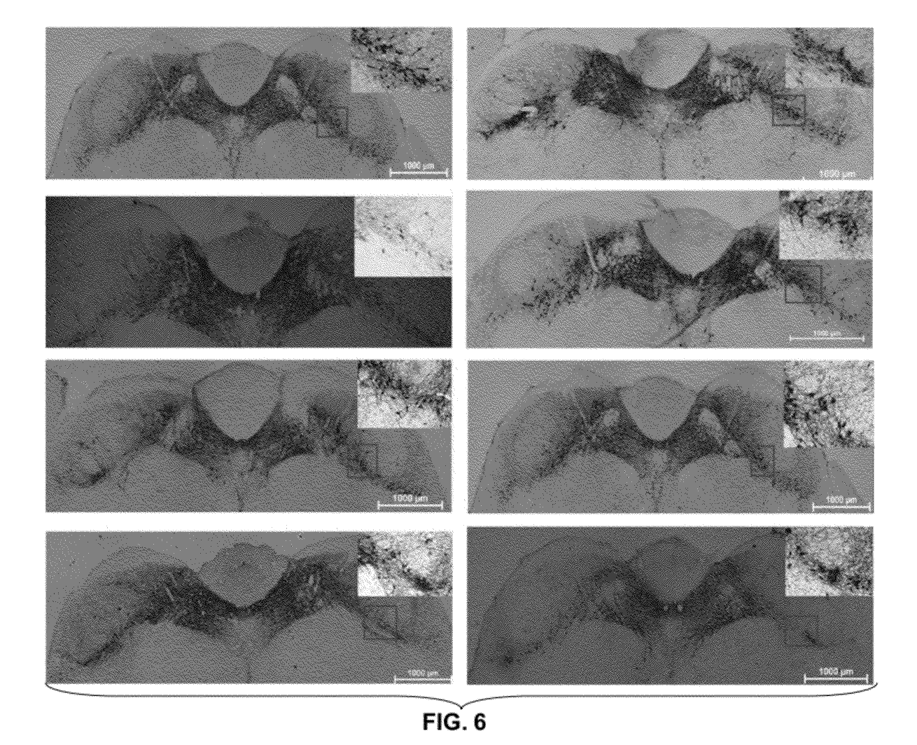

### DETAILED DISCLOSURE OF THE INVENTION

The subject invention provides materials and methods for treating and/or attenuating the progress of Parkinson's Disease (PD) via the administration of a cysteamine compound. In one embodiment, the subject invention provides materials and methods for preventing the onset of PD.

The term “treatment” or any variation thereof (i.e., treat, treating, etc.), as used herein, refers to any treatment of a patient diagnosed with PD. The term treatment, as used herein, includes: (i) ameliorating the symptoms associated with PD in a patient diagnosed with PD; and/or (ii) relieving (such as attenuate the progress of) or remediating PD in a patient diagnosed with PD.

“Parkinson's Disease” or “PD,” as used herein refers to a degenerative disorder of the central nervous system that often impairs the sufferer's motor skills, speech, and other functions. The primary symptoms are the results of decreased stimulation of the motor cortex by the basal ganglia, normally caused by the insufficient formation and action of dopamine, which is produced in the dopaminergic neurons of the brain (specifically the substantia nigra). Secondary symptoms may include high level cognitive dysfunction and subtle language problems.

The identification of those patients who are in need of treatment for PD is well within the knowledge and ability of one skilled in the art. By way of example, a clinician skilled in the art can readily identify, by the use of clinical tests, neurologic and physical examination, and medical/family history, those patients who are suffering from PD as well as those who are predisposed to developing PD and thus readily determine if an individual is a patient in need of treatment for PD.

The term “patient,” as used herein, describes an organism, including mammals, to which treatment with the compositions according to the present invention is provided. Mammalian species that benefit from the disclosed methods of treatment include, but are not limited to, apes, chimpanzees, orangutans, humans, monkeys; and domesticated animals (i.e., pets) such as dogs, cats, mice, rats, guinea pigs, and hamsters.

“Concurrent administration” and “concurrently administering,” as used herein, includes administering a compound or therapeutic method suitable for use with the methods of the invention (administration of a cysteamine compound) in the treatment of PD. In certain embodiments, a cysteamine compound is concurrently administered with an additional therapeutic agent known to be useful in treating PD. For example, according to the subject invention, a cysteamine compound can be concurrently administered with therapeutic methods or agents useful in the treatment of PD (i.e., general wellness maintenance, physiotherapy, exercise, nutrition, administration of agents such as catechol-O-methyl transferase (COMT) inhibitors (such as entacapone, tolcapone, nitecapone); dopamine agonists (such as pramipexole, ropinerole, bromocriptine, and pergolide); amantadine; benztropine; trihexyphenydil; deprenyl; and L-DOPA alone or in combination with carbidopa or benserazide).

In accordance with the subject invention, a therapeutic agent can be provided in admixture with a cysteamine compound, such as in a pharmaceutical composition; or the agent and cysteamine can be provided as separate compounds, such as, for example, separate pharmaceutical compositions administered consecutively, simultaneously, or at different times. Preferably, if the cysteamine compound and the known agent (or therapeutic method) for treating PD are administered separately, they are not administered so distant in time from each other that the cysteamine compound and the known agent cannot interact.

As used herein, reference to a “cysteamine compound” includes cysteamine, the various cysteamine salts, which include pharmaceutically acceptable salts of a cysteamine compound, as well as prodrugs of cysteamine that can, for example, be readily metabolized in the body to produce cysteamine. Also included within the scope of the subject invention are analogs, derivatives, conjugates, and metabolites of cysteamine, which have the ability as, described herein to modulate biological factors in the treatment of a biological condition, prevention of a biological condition in an at-risk patient, or in the treatment of a complication, condition, or disease associated with the biological condition of interest. Various analogs, derivatives, conjugates, and metabolites of cysteamine are well known and readily used by those skilled in the art and include, for example, compounds, compositions and methods of delivery as set forth in U.S. Pat. Nos. 6,521,266; 6,468,522; 5,714,519; and 5,554,655.

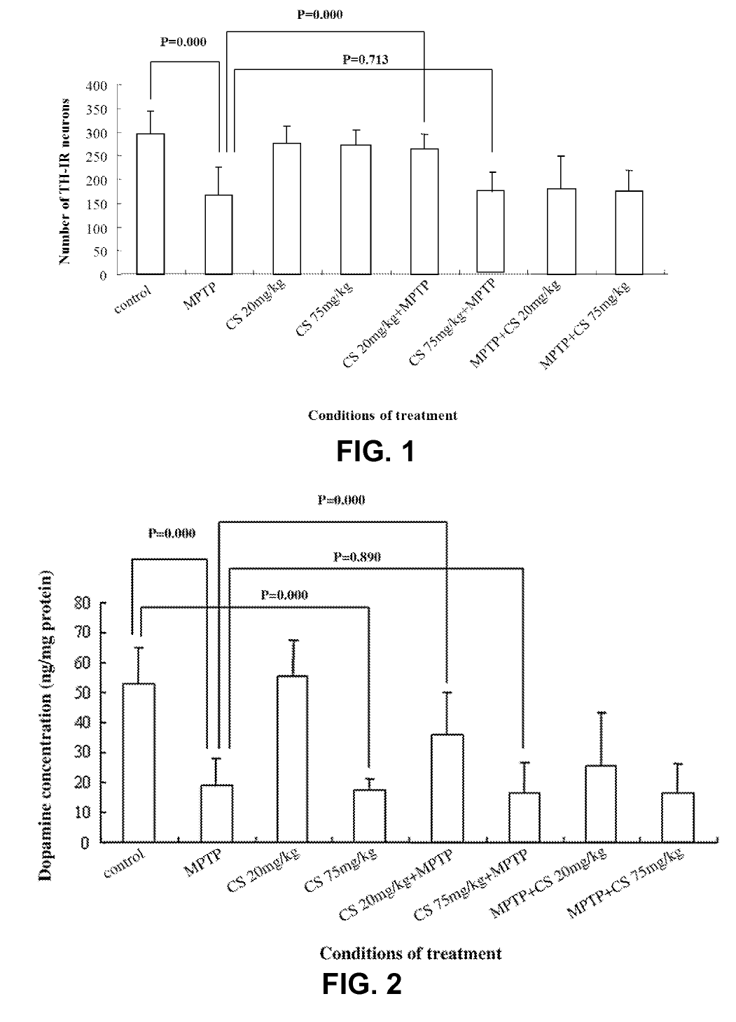

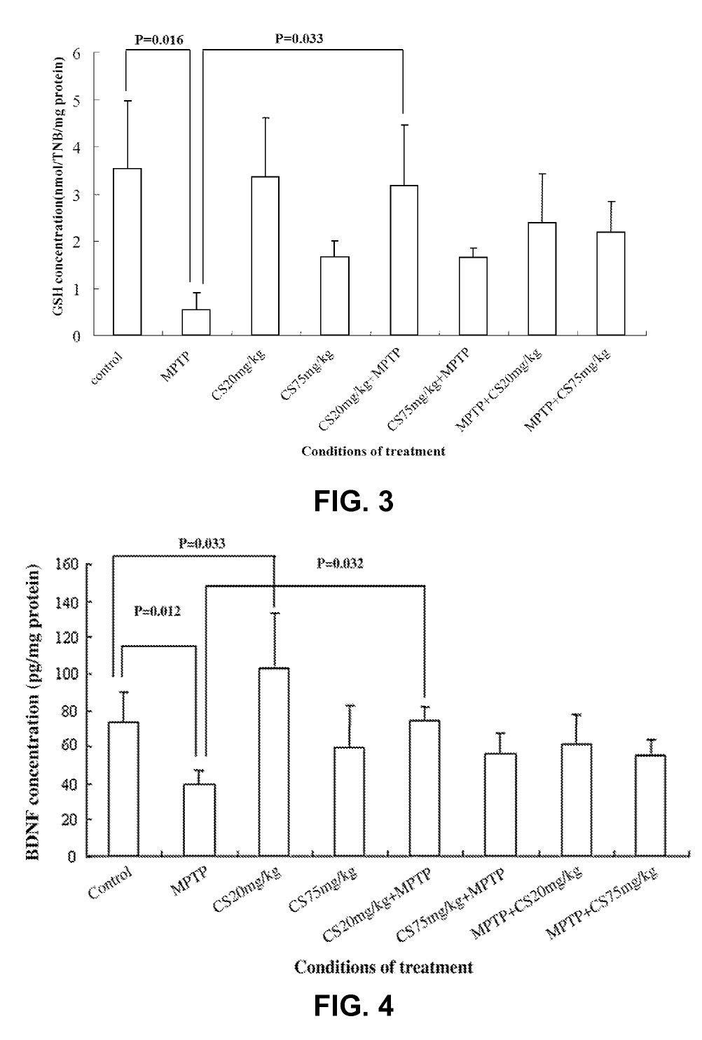

As contemplated herein, a cysteamine compound includes compounds that are known to enhance the endogenous production of cysteamine, including pantothenic acid. Pantothenic acid is a naturally occurring vitamin that is converted in mammals to coenzyme A, a substance vital to many physiological reactions. Cysteamine is a component of coenzyme A, and increasing coenzyme A levels results in increased levels of circulating cysteamine. Alkali metal salts, such as magnesium phosphate tribasic and magnesium sulphite (Epsom salts), enhance formation of coenzyme A. Furthermore, breakdown of coenzyme A to cysteamine is enhanced by the presence of a reducing agent, such as citric acid. Thus, the combination of pantothenic acid and alkali metal salts results in increased coenzyme A production and concomitantly, cysteamine. Accordingly, in one embodiment of the subject invention, the advantages of cysteamine, as set forth herein, can be achieved by promoting the endogenous production of cysteamine through natural metabolic process such as through the action of co-enzyme A or as a metabolite of cysteine (see FIGS. 1 and 2) or administration of pantothenic acid.

The term “pharmaceutically acceptable salt,” as used herein, refers to any salt of a cysteamine compound that is pharmaceutically acceptable and does not greatly reduce or inhibit the activity of the cysteamine compound. Suitable examples include acid addition salts, with an organic or inorganic acid such as acetate, tartrate, trifluoroacetate, lactate, maleate, fumarate, citrate, methane, sulfonate, sulfate, phosphate, nitrate, or chloride. Preferred pharmaceutically acceptable salts of cysteamine also include, but are not limited to, cysteamine hydrochloride. Phosphocysteamine can also be used.

The term “effective amount,” as used herein, refers to the amount necessary to provide an observable effect in at least one biological factor or marker associated with PD (i.e., ameliorating any loss of dopaminergic neurons, ameliorating any reduction in striatal dopamine concentrations, suppressing production of pro-oxidants such as ROS and MDA, attenuating any reduction in glutathione (GSH) levels, and restoring secretion of BDNF by neurons) for use in treating PD. In certain embodiments, the effective amount enables a 5%, 25%, 50%, 75%, 90%, 95%, 99% and 100% attenuation in any loss of dopaminergic neurons, in reduced striatal dopamine concentrations, and/or in reduced GSH levels. In alternate embodiments, the effect amount enables 5%, 25%, 50%, 75%, 90%, 95%, 99% and 100% suppression of pro-oxidants such as ROS and MDA, particularly in dopaminergic neurons. In further embodiments, the effective amount enables 5%, 25%, 50%, 75%, 90%, 95%, 99% and 100% restoration in secretion of BDNF by neurons.

The present invention provides, for the first time, beneficial materials and methods for treating and/or attenuating the progress of PD via the administration of a cysteamine compound. In one embodiment, low dosages of a cysteamine compound are administered to a patient to treat and/or attenuate the progress of PD. More preferably, less than about 50 mg/kg per day of cysteamine or a salt thereof, is administered to a patient to treat and/or attenuate the progress of PD. Even more preferably, up to about 20 mg/kg per day of cysteamine, or a salt thereof, is administered to a patient to treat and/or attenuate the progress of PD.

A cysteamine compound can be administered concurrently with other known agents and/or therapies used to treat PD including, without limitation, general wellness maintenance, physiotherapy, exercise, nutrition, administration of agents such as catechol-O-methyl transferase (COMT) inhibitors (such as entacapone, tolcapone, nitecapone); dopamine agonists (such as pramipexole, ropinerole, bromocriptine, and pergolide); amantadine; benztropine; trihexyphenydil; deprenyl; and L-DOPA alone or in combination with carbidopa or benserazide.

The compositions of the invention can be used in a variety of routes of administration, including, for example, orally-administrable forms such as tablets, capsules or the like, or via parenteral, intravenous, intramuscular, transdermal, buccal, subcutaneous, suppository, or other route. Such compositions are referred to herein generically as “pharmaceutical compositions.” Typically, they can be in unit dosage form, namely, in physically discrete units suitable as unitary dosages for human consumption, each unit containing a predetermined quantity of active ingredient calculated to produce the desired therapeutic effect in association with one or more pharmaceutically acceptable other ingredients, i.e., diluent or carrier.

The cysteamine compounds of the subject invention can be formulated according to known methods for preparing pharmaceutically useful compositions. Formulations are described in a number of sources, which are well known and readily available to those skilled in the art. For example, Remington's Pharmaceutical Science (Martin E W [1995] Easton Pa., Mack Publishing Company, 19^{th} ed.) describes formulations that can be used in connection with the subject invention. Formulations suitable for parenteral administration include, for example, aqueous sterile injection solutions, which may contain antioxidants, buffers, bacteriostats, and solutes, which render the formulation isotonic with the blood of the intended recipient; and aqueous and nonaqueous sterile suspensions, which may include suspending agents and thickening agents. The formulations may be presented in unit-dose or multi-dose containers, for example sealed ampoules and vials, and may be stored in a freeze dried (lyophilized) condition requiring only the condition of the sterile liquid carrier, for example, water for injections, prior to use. Extemporaneous injection solutions and suspensions may be prepared from sterile powder, granules, tablets, etc. It should be understood that in addition to the ingredients particularly mentioned above, the formulations of the subject invention can include other agents conventional in the art having regard to the type of formulation in question.

In one embodiment, compositions comprising a cysteamine compound and a carrier such as inclusion compound host materials are provided. The “inclusion compound host materials” as described herein, interact with the cysteamine compound to increase aqueous solubility, increase chemical stability, and/or enhance drug (such as a cysteamine compound) delivery to and through biological membranes. It is believed that by providing a carrier such as inclusion compound host materials, a stabilized cysteamine compound molecule can be safely delivered to a patient at a dosage that will not induce toxicity. In addition, such carrier materials can include coating materials (i.e., enteric-coatings) that allow dissolution of the coating in an alkaline environment such as in the intestines.

An inclusion compound host material that can be used in accordance with the subject invention includes those disclosed in U.S. Patent Application No. 20040033985, incorporated herein in its entirety. Contemplated inclusion compound host materials include proteins (such as albumin), crown ethers, polyoxyalkylenes, polysiloxanes, zeolites, cholestyramine, colestipol, colesevelam, colestimide, sevelamer, cellulose derivatives, dextran derivatives, starch, starch derivatives, and pharmaceutically acceptable salts thereof. Contemplated cellulose derivatives and dextran derivatives include DEAE-cellulose, guanidinoethylcellulose, or DEAE-Sephadex. Favorable starches or starch derivatives to be included in the compositions of the invention include cyclodextrin, retrograded starch, degraded starch, a combination of retrograded and degraded starch, hydrophobic starch, amylase, starch-diethylaminoethylether, and starch-2-hydroxyethylether.

According to the subject invention, preferred inclusion compound host materials include, but are not limited to, cyclodextrin and/or its derivatives (i.e., methyl β-cyclodextrin (M-β-CD), hydropropyl β-cyclodextrin (HP-β-CD), hydroethyl β-cyclodextrin (HE-β-CD), polycyclodextrin, ethyl β-cyclodextrin (E-β-CD) and branched cyclodextrin. As one skilled in the art will appreciate, any cyclodextrin or mixture of cyclodextrins, cyclodextrin polymers, or modified cyclodextrins can be utilized pursuant to the present invention. Cyclodextrins are available from Wacker Biochem Inc., Adrian, Mich. or Cerestar USA, Hammond, Ind., as well as other vendors. Formation of inclusion complexes using cyclodextrin or its derivatives protects the constituent (i.e., cysteamine compound) from loss of evaporation, from attack by oxygen, acids, visible and ultraviolet light and from intra- and intermolecular reactions.

The general chemical formula of cyclodextrin is (C₆O₅H₉)ₙ. The content of inclusion compound host materials in compositions of the subject invention can range from about 1 to 80 wt %. Preferably, the content of inclusion compound host materials in compositions of the invention range from about 1 to 60 wt %. The actual amount of the inclusion compound host materials used will depend largely upon the actual content of cysteamine compound and therapeutic agents, if any, used in preparing compositions of the invention.

Administration of a cysteamine compound, in accordance with the subject invention, can be accomplished by any suitable method and technique presently or prospectively known to those skilled in the art. In a preferred embodiment, a cysteamine compound is formulated in a patentable and easily consumed oral formulation such as a pill, lozenge, tablet, gum, beverage, etc. The consumption is then taken at, prior to, or after, the diagnosis of Parkinson's Disease.

In accordance with the invention, compositions comprising, as an active ingredient, an effective amount of the cysteamine compound and one or more non-toxic, pharmaceutically acceptable carrier or diluent. Examples of such carriers for use in the invention include ethanol, dimethyl sulfoxide, glycerol, silica, alumina, starch, sorbitol, inosital, xylitol, D-xylose, manniol, powdered cellulose, microcrystalline cellulose, talc, colloidal silicon dioxide, calcium carbonate, magnesium cabonate, calcium phosphate, calcium aluminium silicate, aluminium hydroxide, sodium starch phosphate, lecithin, and equivalent carriers and diluents.

To provide for the administration of such dosages for the desired therapeutic treatment, compositions of the invention for treating and/or attenuating the progress of PD will typically comprise between about 0.1% and 95%, of the total composition including carrier or diluent. The dosage used can be varied based upon the age, weight, health, or the gender of the individual to be treated.

In certain embodiments of the invention, a patient is assessed to identify the risk of developing PD prior to the concurrent administration of a cysteamine compound and at least one additional therapeutic agent (i.e., general wellness maintenance, physiotherapy, exercise, nutrition, administration of agents such as catechol-O-methyl transferase (COMT) inhibitors (such as entacapone, tolcapone, nitecapone); dopamine agonists (such as pramipexole, ropinerole, bromocriptine, and pergolide); amantadine; benztropine; trihexyphenydil; deprenyl; and L-DOPA alone or in combination with carbidopa or benserazide). Various markers have recently been identified as important markers that predate the clinical onset of PD.

Various markers have recently been identified as important markers that predate the clinical onset of PD. For example, assessment of induction of alpha-synuclein expression in skin fibroblasts, mean alpha-synuclein value in cerebral spinal fluid, as well as multianalyte profile (MAP) of tau, BDNF, interleukin 8, Abeta42, beta2-microglobulin, vitamin D binding protein, apolipoprotein (apo)) AII, and apoE, have all been implicated as being effective in identifying PD before disease or symptom manifestation (Graeber, “Biomarkers for Parkinson's Disease,” Exp Neurology, 216(2):2009:249-253 (2009)). Heredity is another factor that can be used, alone or in combination, to determine whether an at-risk patient is predisposed to developing PD. Such methods for identifying at-risk patients for PD can be used in accordance with the subject invention.

In one embodiment, the dosage of a cysteamine compound administered to a patient to treat and/or attenuate the progress of PD is about 1 mg/kg of body weight to about 100 mg/kg of body weight per day. Preferably, cysteamine, or a salt thereof, is administered daily at less than about 50 mg/kg of body weight to treat and/or attenuate the progress of PD. Even more preferably, less than about 20 mg/kg of body weight of cysteamine, or a salt thereof, is administered daily to a patient to treat and/or attenuate the progress of PD.

Following are examples that illustrate procedures for practicing the invention. These examples should not be construed as limiting. All percentages are by weight and all solvent mixture proportions are by volume unless otherwise noted.

### Materials and Methods

### Drugs and Chemicals

1-methyl-4-phenyl-1,2,3,6-tetrahydropyridine hydrochloride (MPTP-HCl), dopamine standard, diaminobenzidine tetrahydrochloride (DAB), 2′,7′-dichlorodihydrofluorescein diacetate (DCDHF-DA), Rabbit anti-tyrosine hydroxylase (TH) affinity purified monoclonal antibody were procured from Sigma-Aldrich. Cysteamine was procured from Walcom-biochem Inc. HPLC-grade acetonitrile and methanol was bought from Fisher Science China. Brain-derived neurotrophic factors (BDNF) was purchased from XiTang Biological Technology (Shanghai, China). Glutathione kit was purchased from Beyotime Institute of Biotechnology (Guangzhou, China). Thiobarbituric acid and superoxide dismutase were obtained from Nanjing Jiancheng Institute of Biological Engineering.

### Animal and Treatment of Cysteamine and MPTP-HCl

Adult male C57/BL mice were purchased from the Animal Center at the Chinese Academy of Sciences in Beijing, and housed five per cage in a light and temperature-controlled room. They were allowed free access to standard food and water ad libitum. All experimental procedures were in accordance with the Guide for the Care and Use of Laboratory Animals (NIH publications Nos. 80-23, revised 1996) and were approved by the institutional animal experimental ethical committee.

C57/BL mice weighted 20-22 g were used. Mice were treated with cysteamine and/or induced by MPTP fresh preparations according to the group assignments. Group assignments (n=8/group) were as follows: (1) saline (control) group, (2) MPTP alone (see below), (3) cysteamine (CS) 20 mg/kg/d alone for two weeks, (4) CS 75 mg/kg/d alone for two weeks, (5) CS 20 mg/kg/d for total 14 days and MPTP was given from day 5 to 9, (6) CS 75 mg/kg/d for total 14 days and MPTP was given from day 5 to 9, (7) simultaneous administration of MPTP and CS 20 mg/kg/d for 5 days followed by CS for additional 9 days, (8) simultaneous administration of MPTP and CS 75 mg/kg/d for 5 days followed by CS for additional 9 days. The control group was injected with 0.9% saline. MPTP (20 mg/kg) was intraperitoneally (i.p.) injected into the mice twice daily at an interval of 12 hrs for the initial 2 days and once per day on day 3-5 (Tremblay et al., “Neuroprotective effects of cystamine in aged parkinsonian mice,” Neurobiol Aging. 27:862-870 (2006)). CS (20 mg/kg/d and 75 mg/kg/day were injected alone for 14 days.

### Tissue Preparation

After treatment for 14 days, the animals were sacrificed by decapitation. Tissues were quickly removed from the brain on a Petri dish placed on ice using laboratory microscope (Chan et al., “Development of a high resolution three-dimensional surgical atlas of the murine head for strains 129S1/SvImJ and C57Bl/6J using magnetic resonance imaging and micro-computed tomography,” Neuroscience. 144:604-615 (2007)). Bilateral corpus striatum stored at −80° C. until used for HPLC analysis. Bilateral substantia nigra were immediately homogenized using an Ultrasonic Cell Disruptor (Biologics, Model 150 V/T), and which were centrifuged. The supernatant was used for the estimation of various enzyme activities.

### Assay of Dopamine (DA) by High Performance Liquid Chromatography (HPLC)

Samples were processed blindly to the treatment conditions for HPLC analysis with electrochemical detector (ECD) using the method by Beyer (Bayer, et al., 2002). Bilaterally corpus striatum tissues were sonicated in 1 ml of pre-colded 0.4 N HClO₄. The resulting homogenates were centrifuged twice for 12 min at 12000×g at 4° C. Mobile phase consisting of 0.035 mol/L anhydrous citric acid, 0.09 mol/L without Water-sodium acetate, 0.23 mmol/L sodium alkane sulfonate, 0.13 mmol/L EDTA, 10% methanol (pH4.7). Electrochemical conditions for the experiment were +0.50V. Separation was carried out at a flow rate of 1.0 ml/min. Samples (20 μl) were automatic injected and were quantified by comparison of the area under the curve (AUC) against reference standards. The concentrations of DA were expressed as ng/mg protein of the brain tissue. Protein content was determined in tissue supernatants by the method of Lowry using bovine serum albumin as a standard (Lowry et al., “Protein measurement with the Folin phenol reagent,” J Biol. Chem. 193:265-275 (1951)).

### Tyrosine Hydroxylase Immunohistochemistry

Twenty-four hours later the last injection, mice were anesthetized with chloral hydrate (100 mg/kg). The mice were subjected to thoracotomy and perfusion with ice-cold 0.9% sodium chloride 50 ml, then with 4% paraformaldehyde (PFA) 100 ml in 0.01 M PBS through the left ventricular. After fixation, the brain was post-fixed and then cryoprotected in 30% sucrose for 24 h. The brains were sectioned through the substantia nigra (SN) coronally at 30 um thickness on a cryostat (Leica CM1900, Nussloch, Germany). Sections were collected in 0.01 M PBS and processed free floating. The free floating sections were washed 3 times in Tris-buffered saline (TBS), incubated with H₂O₂ (3%) for 30 min and blocked with 5% normal goat serum (NGS), 0.2% Triton X-100 in TBS. Sections were incubated for 24 h with primary rabbit anti-TH monoclonal antibody (1:10000) (Watanabe et al., “Protective effects of M, Matsubara, Imai Y, Araki T. neuronal nitric oxide synthase inhibitor in mouse brain against MPTP neurotoxicity: an immunohistological study,” Eur Neuropsychopharmacol. 14:93-104 (2004)). Sections were washed in PBS and incubated for 2 hr at room temperature in secondary antibodies: biotinylated goat anti-rabbit (1:500) diluted in blocking buffer. The sections were incubated for 2 hr at room temperature in horseradish peroxidase labeled avidin work streptomycin solution (1:500). TH immunoreactivity was visualized in SN after incubation in DAB and Nickel sulfate salt for 5-10 min. Sections were mounted on slides, dried, dehydrated in graded ethanol, cleared in xylene, and mounted with DPX mounting medium and coverslip. To test the specificity of the immunostaining, control sections were processed in an identical manner but with the primary antibody omitted. After TH-staining, the image of the sections from the substantia nigra were digitalized with NIS-Elements imaging software (Nikon). The number of neurons was measured in the bilateral substantia nigra.

### Measurement of Oxidative Stress Parameters

The ROS level was measured using 2′,7′-dichlorodihydrofluorescein diacetate (DCDHF-DA). DCDHF-DA which is cell-permeable and non-fluorescent, and, in the presence of ROS it is oxidized inside cells and transformed into a fluorescent compound, 2′,7′-dichlorofluorescein (DCDF), which remains trapped within the cell. To measure the ROS level in substantia nigra, isolated cells (10⁵/ml) were incubated with 50 μM DCDHF-DA for 40 min at 37° C. and washed twice in PBS. The fluorescence was monitored in microplate reader (Liu, et al., 2009). For each sample, 10,000 cells were acquired. Data were expressed as an average of fluorescence intensity of analyzed cell population (mean fluorescence intensity±SD).

The formation of thiobarbituric acid reactive species (TEARS) during an acid-heating reaction was determined by the absorbance at 532 nm. The rate of lipid peroxidation was expressed as nmol/mg protein. SOD activity was detected based on its ability to inhibit the superoxide anion free radical of O²⁻ generated by the xanthine/xanthine oxidase system. The absorbance at 550 nm was monitored and SOD levels were expressed as U/mg protein. The total glutathione (reduced and oxidized form of glutathione) was determined using glutathione reductase. Absorbance was measured at 412 nm using microplate reader. The standard curve was obtained from absorbance of the diluted commercial GSH that was incubated in the mixture as in samples. Results were reported as nmol TNB/mg protein.

### Assay for Brain-Derived Neurotrophic Factor (BDNF)

The concentration of BDNF was measured by an ABC-ELISA method, according to the manufacturer's protocol (Beyotime Institute of Biotechnology). The assay sensitivity was 15 pg/ml and no significant cross reactivity with other related neurotrophins. With BDNF anti-mouse antibody-coated plate in ELISA, BDNF of the standard materials and samples combined with the monoclonal antibody, by adding biotinylated anti-mouse BDNF, the formation of immune complexes connected to the board, horseradish peroxide complex enzyme labeled streptavidin and biotin combination, adding the substrate fluid to show blue, plus the final termination of liquid sulfuric acid, and then measured OD value 450 nm. OD value and BDNF concentration are proportional to the standard curve obtained the standard BDNF concentrations. Data are represented as pg/mg protein.

### Statistical Analysis

Comparison among over two groups was performed by one-way ANOVA with a post hoc test. Differences were considered significant when P<0.05. Data analyses were analyzed by the Statistical Package for the Social Sciences software (SPSS 16.0 for Windows).

### Example 1

### Low Dose of Cysteamine Prevented MPTP-Mediated Loss of TH-IR Neurons in the Substantia Nigra Pars Compacta (SNpc) and Loss of Striatal Dopamine Concentration

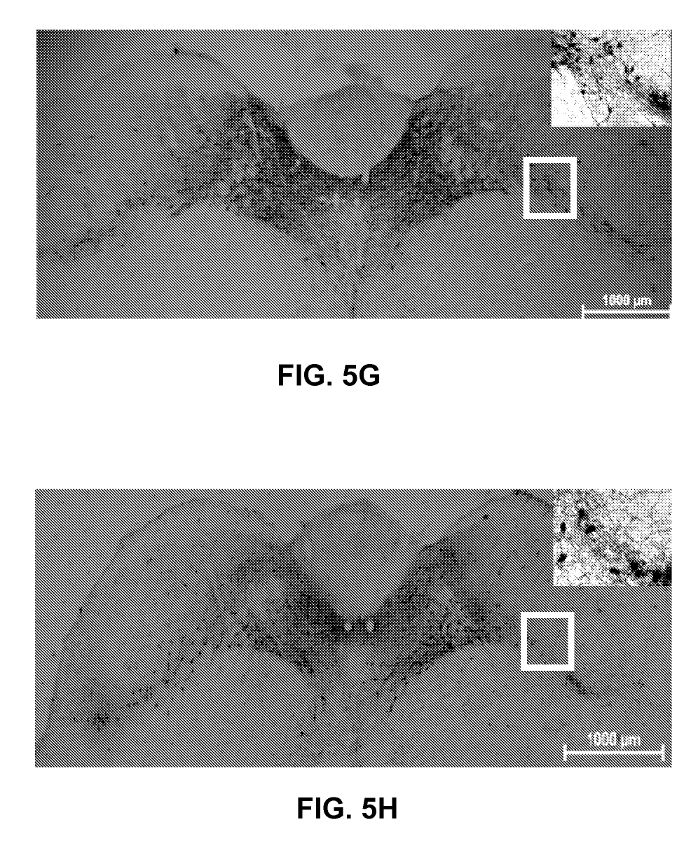

To examine the effect of cysteamine on the number of DAnergic neurons, histochemistry analysis was performed in cells derived from the substantia nigra pars compacta (SNpc) using anti-TH monoclonal antibody. As shown in FIGS. 1 and 5A-H, the number of TH-immunoresponsive (TH-IR) nigral neurons significantly decreased in the MPTP-induced mice (166.65±58.15) (FIGS. 1 and 5B), as compared with control mice (295.65±47.47) (P=0.000) (FIGS. 1 and 5A). However, the loss of TH-IR neurons from mice injected with cysteamine (20 mg/kg/d) (FIGS. 1 and 5C) 4 days prior to and subsequently along with MPTP-treatment (CS20 mg/kg/d+MPTP) was ameliorated (262.45±32.75) to a significant extent (P=0.000, compared with MPTP alone) (FIGS. 1 and 5E).

Interestingly, the higher dose (75 mg/kg/d) of cysteamine (CS75 mg/kg/d+MPTP) did not attenuate the loss of number (172.10±42.69) of TH-IR neurons (compared with MPTP alone, P=0.713) (FIGS. 1 and 5F) neither high dose of cysteamine alone (271.05±44.93) as compared to control (P=0.110) (FIGS. 1 and 5D). However, simultaneous treatment of cysteamine at either low or high dose with MPTP (MPTP+CS20 mg/kg/d or MPTP+CS75 mg/kg/d) did not suppress the reduction of number of TH-IR neurons (179.5±68.11 and 174.05±45.78, respectively) induced by MPTP (both P>0.05, compared with MPTP alone) (FIGS. 1 and 5G-5H).

Similarly, the striatal DA concentration (ng/mg protein) of MPTP-treated mice (26.38±6.47) was significantly decreased as compared with control (57.62±12.70) (P=0.000). Pretreatment with low dose of cysteamine (20 mg/kg/d) daily for 4 days before and along with MPTP significantly protected against MPTP-induced striatal DA depletion (47.96±10.57) (P=0.000). But pretreatment with high dose of cysteamine (75 mg/kg/d) did not attenuate the loss of striatal DA concentration (25.65±7.71) (P=0.89, compared with MPTP alone) and the treatment with high dose of cysteamine alone (CS 75 mg/kg/day) even significantly decreased the DA concentration (32.22±4.71) (P=0.000, compared with non-treated control). Simultaneous treatment with cysteamine (20 mg/kg/d or 75 mg/kg/day) during MPTP-treatment did not ameliorate MPTP-induced striatal DA depletion (36.27±16.42 and 25.08±8.10, respectively) (FIG. 2).

### Example 2

### Cysteamine Decreases the Concentrations of ROS and MDA While Increases the Concentration of GSH in the SNpc of MPTP-Treated Mice

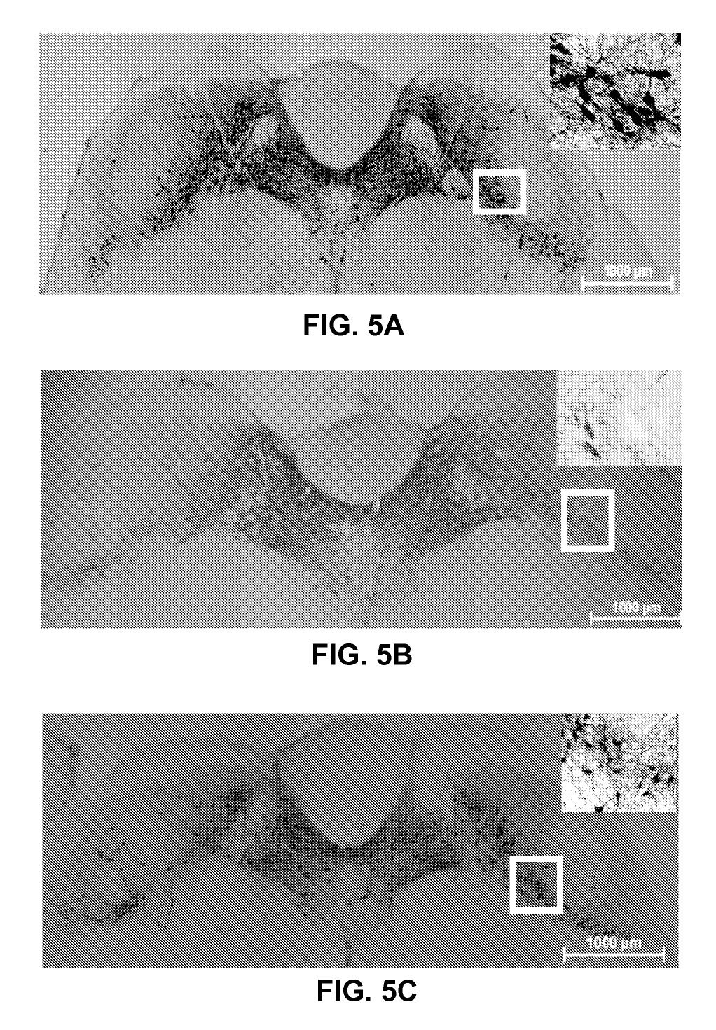

To examine the effects of cysteamine on oxidative-stress conditions, both pro- and anti-oxidants were measured in dopaminergic neurons derived from MPTP-treated mice. As shown in Table 1, the levels of both malondialdehyde (MDA) and reactive oxygen species (ROS) were significantly elevated after MPTP exposure (MDA: P=0.0001; ROS: P=0.005). These decreases were suppressed by pretreatment with both high (MDA: P=0.005; ROS: P=0.04) and low dose of cysteamine (MDA: P=0.001; ROS: P=0.04), respectively. In contrast, activity of superoxide dismutase (SOD), the scavenger for H₂O₂, was not significantly changed. Level of GSH (nmol TNB/mg protein) was significantly decreased in the SNpc of the MPTP-treated mice (0.55±0.36) as compared with control group (3.52±1.44) (P=0.016), which was partially restored (3.16±1.28) by pretreatment with the low dose of cysteamine (P=0.033) but not the high dose group (P=0.356) (FIG. 3).

TABLE 1
Effect of cysteamine on malondialdehyde (MDA), reactive oxygen species (ROS), and superoxide
dismutase (SOD) in the substantia nigra of MPTP-lesion mice (n = 8)

| Species/ | | | | | C20 mg + |
|---|---|---|---|---|---|
| Enzymes | Control | MPTP | C20 mg | C75 mg | MPTP |
| MDA | 7.89 ± 0.65 | 10.84 ± 2.65* | 8.82 ± 2.44 | 8.90 ± 2.14 | 8.25 ± 0.90^{#} |
| (nmol/mg) | | | | | |
| ROS | 13010 ± 261 | 20207 ± 203* | 17460 ± 779* | 17980 ± 419* | 17411 ± 589*, ** |
| (MFI) | | | | | |
| SOD | 52.55 ± 13.35 | 57.73 ± 9.57 | 58.88 ± 17.66 | 59.13 ± 10.66 | 62.11 ± 4.74 |
| (U/mg) | | | | | |

| Species/ | C75 mg + | MPTP + | MPTP + |
|---|---|---|---|
| Enzymes | MPTP | C20 mg | C75 mg |
| MDA | 8.56 ± 1.08^{#} | 9.86 ± 0.91** | 9.51 ± 0.57** |
| (nmol/mg) | | | |
| ROS | 17411 ± 362*, ** | 19539 ± 431* | 21976 ± 489* |
| (MFI) | | | |
| SOD | 58.53 ± 5.82 | 66.77 ± 4.64 | 59.58 ± 2.24 |
| (U/mg) | | | |

MDA: *p < 0.05 compared with the control, **p < 0.01 compared with MPTP alone, ^{#}p < 0.05 compared with MPTP alone.
ROS: *p < 0.01 compared with the control, **p < 0.01 compared with MPTP alone, ^{#}p < 0.05 compared with MPTP alone.
SOD: no significant difference compared with each other.

### Example 3

### Cysteamine Increases BDNF Secretion in the SNpc of MPTP-Induced Mice

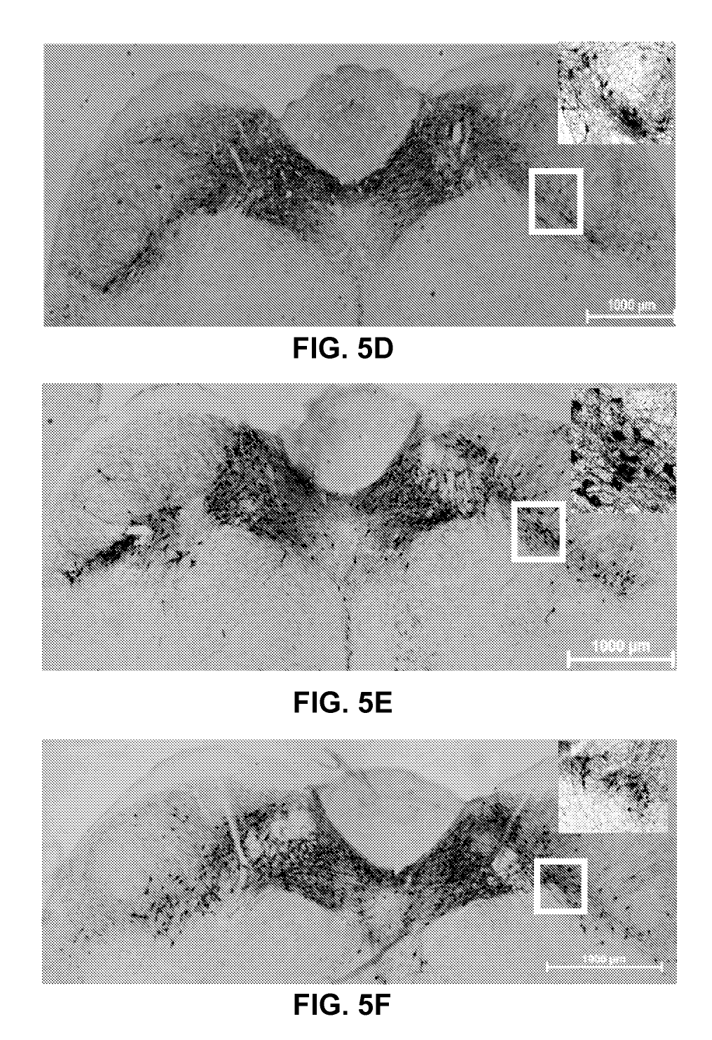

To examine whether cysteamine can affect production of brain-derived neurotrophic factor (BDNF), the level of BDNF protein concentration of BDNF was measured in the substantia nigra by ELISA. As shown in FIG. 4, MPTP-treatment resulted in a significant reduction of BDNF secretion (39.5517.82) as compared to control (78.34116.09) (P=0.016). This reduction was attenuated by low dose of cysteamine (74.2217.79, P=0.014 as compared to MPTP-treated mice), but not high dose (55.43112.15, P=0.242). Interestingly, treatment with low dose of cysteamine alone also stimulated an augmentation of BDNF secretion (103.36130.48) (P=0.033, compared to control). In contrast, simultaneously administration of cysteamine with MPTP did not reverse the decrease of BDNF by MPTP (both P>0.05).

The results from this Example showed that a low dose of cysteamine (20 mg/kg/d) confers a protective effect against MPTP-induced loss of DA neurons, but the relatively high dose (75 mg/kg/d) does not.

It should be understood that the examples and embodiments described herein are for illustrative purposes only and that various modifications or changes in light thereof will be suggested to persons skilled in the art and are to be included within the spirit and purview of this application. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

I claim:

1. A method for attenuating or treating Parkinson's Disease, wherein said method comprises diagnosing a patient with Parkinson's Disease; and administering to the patient an effective amount of a cysteamine compound or pharmaceutically acceptable salt thereof.

2. The method according to claim 1, wherein said cysteamine salt is cysteamine hydrochloride.

3. The method according to claim 1, which comprises administering to the patient daily about 0.1 mg to about 1,000 mg of the cysteamine compound, or pharmaceutically acceptable salt thereof, per kg of the patient body weight.

4. The method according to claim 1, wherein said cysteamine compound is administered orally, parenterally, intravenously, intramuscularly, intraperitoneally, transdermally, via buccal route, subcutaneously, or via suppository.

5. The method according to claim 1, further comprising the step of concurrently administering, with the cysteamine compound, or pharmaceutically acceptable salt thereof, another agent selected from the group consisting of: catechol-O-methyl transferase inhibitors; dopamine agonists; amantadine; benztropine; trihexyphenydil; deprenyl; and L-DOPA alone or in combination with carbidopa or benserazide.

6. A method for treating a disorder associated with a loss of dopaminergic neurons and/or a decrease in dopamine concentration, said method comprising administering, to a patient diagnosed with the disorder, an effective amount of a cysteamine compound, or a pharmaceutically acceptable salt thereof, wherein the administration of the cysteamine compound, or pharmaceutically acceptable salt thereof, results in ameliorating the loss of dopaminergic neurons and/or the decrease in dopamine concentration in the patient.

7. The method of claim 6, wherein the dopaminergic neurons are those located in the substantia nigra.

8. The method of claim 6, wherein the decrease in dopamine concentration is in the patient striata.

9. The method according to claim 6, wherein said pharmaceutically acceptable salt is cysteamine hydrochloride.

10. The method according to claim 6, which comprises administering to the patient daily about 0.1 mg to about 1,000 mg of the cysteamine compound, or pharmaceutically acceptable salt thereof, per kg of the patient body weight.

11. A method for treating a disorder associated with an increased production of pro-oxidants, said method comprising administering, to a patient diagnosed with the disorder, an effective amount of a cysteamine compound, or a pharmaceutically acceptable salt thereof; wherein the administration of the cysteamine compound, or pharmaceutically acceptable salt thereof, results in suppressing the production of the pro-oxidants in the patient.

12. The method of claim 11, wherein the pro-oxidants are reactive oxygen species and/or malondialdehyde.

13. The method according to claim 11, wherein said pharmaceutically acceptable salt is cysteamine hydrochloride.

14. The method according to claim 11, which comprises administering to the patient daily about 0.1 mg to about 1,000 mg of the cysteamine compound, or pharmaceutically acceptable salt thereof, per kg of the patient body weight.

15. A method for treating a disorder associated with reduced neuron secretion of a brain-derived neurotrophic factor, said method comprising administering, to a patient diagnosed with the disorder, an effective amount of a cysteamine compound, or a pharmaceutically acceptable salt thereof; wherein the administration of the cysteamine compound, or pharmaceutically acceptable salt thereof, results in restoring neuron secretion of the brain-derived neurotrophic factor in the patient.

16. The method of claim 15, wherein the neurons are those from the substantia nigra pars compact and the brain-derived neurotrophic factor is glutathione.

17. The method according to claim 15, wherein said pharmaceutically acceptable salt is cysteamine hydrochloride.

18. The method according to claim 15 which comprises administering to the patient daily about 0.1 mg to about 1,000 mg of the cysteamine compound, or pharmaceutically acceptable salt thereof, per kg of the patient body weight.

19. A composition comprising an effective amount of a cysteamine compound, or pharmaceutically effective salt thereof, and another agent used to treat Parkinson's Disease.

20. The composition according to claim 19, wherein the agent is selected from the group consisting of: catechol-O-methyl transferase inhibitors; dopamine agonists; amantadine; benztropine; trihexyphenydil; deprenyl; and L-DOPA alone or in combination with carbidopa or benserazide.
